SEQUENCE LISTING (14 sequences)

SEQ ID NO: 1 - protein, 15 aa, Artificial Sequence
  Synthetic
GGGGSGGGGSGGGGS

SEQ ID NO: 2 - protein, 20 aa, Artificial Sequence
  Synthetic
GGGGSGGGGSGGGGSGGGGS

SEQ ID NO: 3 - protein, 25 aa, Artificial Sequence
  Synthetic
GGGGSGGGGSGGGGSGGGGSGGGGS

SEQ ID NO: 4 - protein, 30 aa, Artificial Sequence
  Synthetic
GGGGSGGGGSGGGGSGGGGSGGGGSGGGGS

SEQ ID NO: 5 - protein, 35 aa, Artificial Sequence
  Synthetic
GGGGSGGGGSGGGGSGGGGSGGGGSGGGGSGGGGS

SEQ ID NO: 6 - protein, 40 aa, Artificial Sequence
  Synthetic
GGGGSGGGGSGGGGSGGGGSGGGGSGGGGSGGGGSGGGGS

SEQ ID NO: 7 - protein, 45 aa, Artificial Sequence
  Synthetic
GGGGSGGGGSGGGGSGGGGSGGGGSGGGGSGGGGSGGGGSGGGGS

SEQ ID NO: 8 - protein, 50 aa, Artificial Sequence
  Synthetic
GGGGSGGGGSGGGGSGGGGSGGGGSGGGGSGGGGSGGGGSGGGGSGGGGS

SEQ ID NO: 9 - protein, 55 aa, Artificial Sequence
  Synthetic
GGGGSGGGGSGGGGSGGGGSGGGGSGGGGSGGGGSGGGGSGGGGSGGGGSGGGGS

SEQ ID NO: 10 - protein, 5 aa, Artificial Sequence
  Synthetic
GGGGS

SEQ ID NO: 11 - protein, 9 aa, Artificial Sequence
  Synthetic
GGSGGSGGS

SEQ ID NO: 12 - protein, 60 aa, Artificial Sequence
  Synthetic
GGGGSGGGGSGGGGSGGGGSGGGGSGGGGSGGGGSGGGGSGGGGSGGGGSGGGGSGGGGS

SEQ ID NO: 13 - protein, 60 aa, Artificial Sequence; identical to SEQ ID NO: 12
  Synthetic

SEQ ID NO: 14 - protein, 10 aa, Artificial Sequence
  Synthetic
GGGGSGGGGS

(19) United States
(12) Patent Application Publication
(10) Pub. No.: US 2020/0291115 A1
(43) Pub. Date: Sep. 17, 2020

(54) COMPOSITIONS AND METHODS FOR TREATING DISEASES

(71) Applicants: THE BROAD INSTITUTE, INC., Cambridge, MA (US); MASSACHUSETTS INSTITUTE OF TECHNOLOGY, Cambridge, MA (US)

(72) Inventor: Feng Zhang, Cambridge, MA (US)

(73) Assignees: THE BROAD INSTITUTE, INC., Cambridge, MA (US); MASSACHUSETTS INSTITUTE OF TECHNOLOGY, Cambridge, MA (US)

(21) Appl. No.: 16/819,778

(22) Filed: Mar. 16, 2020

Related U.S. Application Data
(60) Provisional application No. 62/819,093, filed on Mar. 15, 2019.

(51) Int. Cl. C07K 16/28; C07K 16/44; A61K 35/17

(52) U.S. Cl. CPC C07K 16/2809; C07K 16/44; C07K 2319/33; C07K 2317/622; C07K 2317/55; A61K 35/17

(57) ABSTRACT

An engineered, non-naturally occurring molecule for target immunotherapy, comprising: (a) a first binding component capable of binding to a T cell; and (b) a second binding component capable of binding to a diseased cell.

### CROSS-REFERENCE TO RELATED APPLICATIONS

This application claims the benefit of U.S. Provisional Application No. 62/819,093, filed Mar. 15, 2019. The entire contents of the above-identified application are hereby fully incorporated herein by reference.

### STATEMENT REGARDING FEDERALLY SPONSORED RESEARCH

This invention was made with government support under Grant Nos. MH100706 and MH110049 awarded by the National Institutes of Health. The government has certain rights in the invention.

### REFERENCE TO AN ELECTRONIC SEQUENCE LISTING

The contents of the electronic sequence listing (“BROD-2455US_ST25.txt”; Size is 5,626 bytes and it was created on Mar. 16, 2020) is herein incorporated by reference in its entirety.

### TECHNICAL FIELD

The subject matter disclosed herein is generally directed to engineered molecules for treating diseases.

### BACKGROUND

Immunotherapy has the potential to transform treatment for multiple classes of diseases, including cancer and autoimmune disease. The body's natural capacity for surveillance and targeted destruction of cells can be harnessed to selectively eliminate either uncontrollably growing cancer cells or aberrantly regulated autoimmune cells. Cancer immunotherapies typically fall under two types of modalities: 1) targeting checkpoint blockades or 2) recruiting immune cells or complement system to induce tumor lysis. While the former treatment has had success, it relies upon the body's natural tendency to clear cancer cells and may be easily generalized to other cell types. The latter approach of recruiting immune machinery is poised to work for any patient by personalizing it to the molecular makeup of a patient's tumor or antigens involved in their autoimmune disease. Three targeted immune therapies for cell lysis are currently under clinical trials: antibody based therapies, chimeric antigen receptor T-cell (CART) therapies, and bi-specific T-cell engager (BITE) or bi-specific monoclonal antibody therapies. Antibody-only based immunotherapies are often too broad—for example, by targeting all B cells with CD20 receptor in B cell acute lymphocytic leukemia—causing many side effects, such as immunosuppression. CART therapies have been shown to be very effective for treatment of select blood cancers, but they require personalized ex vivo treatment of patient cells or maintenance of stocks of generalized cells, which can be cost-prohibitive relative to antibody-based therapies. BITEs have shown promise in cancer, but similar approaches have not been extended to other potential diseases.

There are currently no immunotherapies that have worked for patients with autoimmune disease. Further, there are many limitations to current cancer immunotherapies, including lack of response in many patients and harsh side effects. Previous studies for leveraging targeted immunotherapies for autoimmune disease rely on either chimeric autoantigen receptor T cells (CAART) to ablate autoantibody producing B-cells or CART Treg cells to reduce immune responses. Since both of these strategies rely on cell therapies, they are more costly and time-intensive than a protein based therapeutic.

### SUMMARY

In one aspect, the present disclosure provides an engineered, non-naturally occurring molecule, comprising (a) a first binding component capable of binding to a T cell; and (b) a second binding component capable of binding to a diseased cell.

In some embodiments, the first and the second binding components are selected from the group consisting of Fab fragment, single-chain variable fragment (scFv), nanobody, aptamer, antigen, and antigen-binding region. In some embodiments, the first and the second binding components are Fab fragments recognizing different antigens. In some embodiments, the method further comprises an Fc region that binds to Fc-gamma receptor positive cells. In some embodiments, the Fc-gamma receptor positive cells are macrophages, neutrophils, eosinophils, dendritic cells, or natural killer cells. In some embodiments, the first and the second binding components are scFvs and linked by a linker. In some embodiments, the first and the second binding components are nanobodies and linked by a linker.

In some embodiments, the first binding component is a Fab fragment and the second binding component is an aptamer or a 10th type III fibronectin (Fn3) domain. In some embodiments, the first binding component is a Fab fragment and the second binding component is an antigen or a fragment thereof. In some embodiments, the antigen is recognized by an autoantibody. In some embodiments, the antigen is selected from the group consisting of tissue transglutaminase, thyroid peroxidase, TSH receptor, mitochondrial antigen, rheumatoid factor, cycle citrullinated peptide, centromere antigen, topoisomerase I, Ro and La antigens, RNP, Sm, dsDNA, cardiolipin, insulin, glutamic acid decarboxylase, tyrosine phosphatase-like protein, platelet integrin non-collagenous domain of basement membrane collagen type IV, desmoglein 1, desmolgein 3, Streptococcal cell-wall antigen, type XVII collagen, dystonin, myelin basic protein, U1-RNP, GM1, GD1a, GT1a, GQ1b, GD3, acetylcholine receptor, and AQP4.

In some embodiments, the T cell is a CD8+ T cell. In some embodiments, the T cell is a CD4+ T cell. In some embodiments, the diseased cell is a tumor cell. In some embodiments, the diseased cell is an autoimmune B cell.

In another aspect, the present disclosure includes a pharmaceutical composition comprising an engineered, non-naturally occurring molecule herein. In some embodiments, the pharmaceutical composition further comprises a pharmaceutically acceptable carrier or excipient.

In another aspect, the present disclosure includes a method of treating a disease, comprising administering a pharmaceutically effective amount of an engineered, non-naturally occurring molecule herein to a subject in need thereof. In some embodiments, the disease is a cancer. In some embodiments, the cancer is selected from the group consisting of melanoma and metastatic cholangiocarcinoma. In some embodiments, the disease is an autoimmune disease.

In some embodiments, the autoimmune disease is selected from the group consisting of celiac disease, Hashimoto's thyroiditis, Graves' disease, primary biliary cirrhosis, rheumatoid arthritis, scleroderma, Sjogren's syndrome, SLE, type I diabetes, autoimmune thrombocytopenic pupura, Goodpasture's syndrome, Pemphigus vulgaris, acute rheumatic fever, bullous pemphigoid, multiple sclerosis, mixed connective tissue disease, Guillain-Barre syndrome, myasthenia gravis, and neuromyelitis optica. In some embodiments, the method further comprises administering to the subject an engineered T cell. In some embodiments, the engineered, non-naturally occurring molecule is capable of binding to the engineered T cell.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—shows examples of bridging molecule designs, including trifunctional antibodies, two component-bridging molecules, aptamer-based bridging molecules, and antigen fusion bridging molecules.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “bind” includes any physical or chemical attachment or close association, which may be permanent or temporary. Generally, an interaction of ionic bonds, hydrogen bonding, hydrophobic forces, van der Waals forces etc., facilitates physical attachment between the ligand molecule of interest and the receptor. The “binding” interaction may be brief, as in the situation where binding causes a chemical reaction to occur. This is typical when the binding component is an enzyme and the analyte is a substrate for the enzyme. In addition, chemical coupling may be a permanent or reversible binding. Reactions resulting from contact between the binding component and the analyte are within the definition of binding for the purposes of the present invention. Binding may be specific. Binding may be reversible, particularly under different conditions. The term “bind” also encompasses the ability of binding. A cell that binds to a target molecule or cell may also mean the cell is capable of binding to the target molecule or cell. In some cases, the term “target” refers to binding or being capable of binding.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides for methods and compositions that are treatments personalized to the molecular makeup of a patent's disease. In some cases, the treatments include engineering precise interactions between a patient's native immune system and antigens involved in the patient's disease. This strategy allows for administering immunotherapy without the need of cell transplantation or ex vivo modification of patient cells.

In general, the present disclosure includes bridging molecules and methods of using these molecules in treating diseases. In some embodiments, the bridging molecules include an engineered, non-naturally occurring molecule for target immunotherapy. The molecule may comprise two or more binding components. One of the binding components may bind to a diseased cell. Another binding component may bind to an immune cell that can destruct the diseased cell (e.g., lysing, or inhibiting the function or activity of the diseased cell). Some examples provided herein include an engineered, non-naturally occurring molecule for target immunotherapy, comprising (a) a first binding component capable of binding to a T cell; and (b) a second binding component capable of binding to a diseased cell.

### Binding Components

A bridging molecule may comprise two or more binding components. A binding component may be any molecule or a portion thereof that binds or is capable of binding to another molecule. In certain examples, a binding component may be a fragment of a bigger molecule. The fragment may bind or be capable of binding to another molecule or cell. The binding between a binding component and its target molecule may be specific.

In some cases, a binding component is a nucleic acid. The nucleic acid may be capable of binding to another nucleic acid, protein, lipid, or other types of molecule. A nucleic acid may be DNA, RNA, or a hybrid thereof, including without limitation, cDNA, mRNA, genomic DNA, mitochondrial DNA, sgRNA, siRNA, shRNA, miRNA, tRNA, rRNA, snRNA, lncRNA, and synthetic (such as chemically synthesized) DNA or RNA or hybrids thereof. In some examples, a nucleic acid is mRNA. The nucleic acid may be double-stranded or single-stranded. Where single-stranded, the nucleic acid may be the sense strand or the antisense strand. Nucleic acids can include natural nucleotides (such as A, T/U, C, and G), modified nucleotides, analogs of natural nucleotides, such as labeled nucleotides, or any combination thereof.

In certain cases, a binding component is a polypeptide or protein. The term “polypeptide” is used interchangeably with the term “protein” and in its broadest sense refers to a compound of two or more subunit amino acids, amino acid analogs, or peptidomimetics. The subunits can be linked by peptide bonds. In another embodiment, the subunit may be linked by other bonds, e.g., ester, ether, etc. Insofar a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically, or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

### Antibodies

In some cases, a binding component may be an antibody or a fragment thereof. In some examples, an antibody may comprise more than one binding component.

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). In some cases, a binding component may be a fragment of an antibody, e.g., a part or portion of an antibody or antibody chain comprising fewer amino acid residues than an intact or complete antibody or antibody chain. Fragments may be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antibody” encompasses any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, 1 gM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

A region of an antibody may also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

A bridging molecule may comprise an antibody-like protein scaffold. One or more binding components may be a part or linked to such scaffold. The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Examples of such scaffolds include those reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of a molecule or a binding component means that the molecule or binding component exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M-1 (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant cross-reactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope, for example, will not significantly cross-react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

A bridging molecule or binding components may be a monoclonal antibody. As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

A binding component may comprise or may be the binding portion of an antibody. The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

The antibodies herein may be humanized antibodies. “Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404097; International Patent Publication No. WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8(10):1057-62 (1995); and U.S. Pat. No. 5,641,870).

In some cases, the antibodies may be blocking antibodies. As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt complement interactions either partially or fully.

The dose of antibody needed in humans to be effective in the treatment cancer differs with the type and severity of the cancer to be treated, the age and condition of the patient, etc. Typical doses of antibody to be administered are in the range of 1 μg to 1 g, preferably 1-1000 μg more preferably 2-500, even more preferably 5-50, most preferably 10-20 m per unit dosage form. In certain embodiments, infusion of antibodies of the present invention may range from 10-500 mg/m².

### Antibody Fragments and Variants

In some embodiments, a binding component may be an antigen-binding fragment or a portion thereof. The term “antigen-binding fragment (Fab)” refers to a polypeptide fragment of an immunoglobulin or antibody that binds antigen or competes with intact antibody (e.g., with the intact antibody from which they were derived) for antigen binding (e.g., specific binding). A binding component may be a Fab on an antibody. In certain cases, a binding component may be a Fab separated or derived from an antibody. A Fab may be composed of one constant and one variable domain of each of the heavy and the light chain. The variable domain may contain the paratope (the antigen-binding site), comprising a set of complementarity determining regions, at the amino terminal end of the monomer.

In certain cases, the binding component comprises antibody fragments that are more simple than a Fab. For example, a binding component may be a variable region (or a portion thereof) of an antibody. In some cases, a binding component is a variable region of an antibody. A variable region may be a portion of antibody heavy chains or light chains that differ in sequence among antibodies and that cooperate in the binding and specificity of each particular antibody for its antigen. Variability is not usually evenly distributed throughout antibody variable regions. It is typically concentrated within three segments of a variable region called complementarity-determining regions (CDRs) or hypervariable regions, both in the light chain and the heavy chain variable regions. The more highly conserved portions of the variable regions are called the framework regions. The variable regions of heavy and light chains comprise four framework regions, largely adopting a beta-sheet configuration, with each framework region connected by the three CDRs, which form loops connecting the beta-sheet structure, and in some cases forming part of the beta-sheet structure. The CDRs in each chain are held in close proximity by the framework regions and, with the CDRs from the other chain, contribute to the formation of the antigen binding site of antibodies. In some examples, variable regions are with Ig-derived antigen-interaction comprises fragments and derivatives of (poly)peptides which at least comprise one CDR derived from an antibody, antibody fragment or derivative thereof. In some cases, the binding component comprises an antigen-binding region.

In certain examples, a binding component is a nanobody. A nanobody (also known as single-domain antibody) refers to an antibody fragment including a single variable domain of an antibody as a monomer form and has characteristics of selectively binding to a specific antigen similarly to an antibody having an intact structure. The molecular weight of the nanobody may be about 12 kDa to about 15 kDa, which is very little when compared to the normal molecular weight (about 150 kDa or about 160 kDa) of an intact antibody (including two heavy chains and two light chains), and in some cases it is smaller than an Fab fragment or scFv fragment.

In one example, a bridging molecule comprises two variable chain fragments or nanobodies fused together. This may yield a smaller bridging molecule (e.g., compared to a full-length antibody). One fragment/nanobody may target a receptor, like CD3, to attach to CD8+ T cells while the other would target the antigen on a diseased cell.

### Aptamers

In certain examples, the binding component may be an aptamer. Aptamers are nucleic acid species that have been engineered through repeated rounds of in vitro selection or equivalently, SELEX (systematic evolution of ligands by exponential enrichment) to bind to various molecular targets, such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers may be capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drive affinity and specificity in antibody-antigen complexes.

### Other Types of Binding Components

The binding components may be other types of molecules that are binding another molecule. For examples, the binding components may be antigens, ligands (ligands of cell receptors), lipid-binding proteins, carbohydrate-binding proteins, fragments thereof, variants thereof, or hybrids thereof. In some cases, binding components may be any protein domain that are capable of interacting with another molecule (e.g., another protein domain). In certain examples, a binding component may be a fibronectin (Fn3) domain (e.g., 10th type III fibronectin (Fn3) domain). A binding component may also be small molecule that specifically bind to cells. In certain examples, a binding component may be a substance (e.g., a solid support such as beads) that specifically bind to certain type(s) of a cells resulting from physical features such as charges or hydrophobicity.

### Example Bridging Molecule Designs

A bridging molecule may comprise multiple binding components. The multiple binding components may be different portions of a bridging molecule. In general, a bridging molecule may be any molecules that are capable of binding to two molecules or cells.

In some embodiments, a bridging molecule is an antibody that has multiple specificities. For example, the antibody may have a Fab (or a portion e.g., a variable region thereof) that binds to a therapeutic cell and another Fab (or a portion e.g., a variable region thereof) that binds to a diseased cell. The antibody (e.g., through fragment crystallizable region (Fc region)) may also bind (thus recruit) another molecule or cell that regulate (e.g., facilitates or enhance) the function of the therapeutic cell.

In one example, an antibody has multiple specificities built into the Fab region. Each variable Fab arm may target a different antigen. For instance, one Fab may target an antigen on a cancer cell while the other Fab may bind an antigen on a cytotoxic T cell such as CD3. The Fc region may then recruit other accessory immune cells, such as natural killer cells to cause cell lysis through antibody-dependent cell-mediated cytotoxicity (ADCC). The value of this approach is the tri-functional nature that involves both T cell- and accessory cell-mediated cytotoxicity. While this example recruits a cytotoxic T cell, it is to note that there may also be benefit to recruiting CD4+ helper T cells, which may help boost the native immune response in the tumor or disease microenvironment. In some cases, the Fc region binds to Fc-gamma receptor positive cells. The Fc-gamma receptor positive cells may be macrophages, nuetrophils, eosinophils, dendritic cells, natural killer cells, or any combination thereof.

In some embodiments, a bridging molecule comprise two binding components linked or fused together. For example, a bridging molecule may comprise one or more fragments of an antibody, such as a nanobody, Fab, Fab′, (Fab′)2, Fv, ScFv, diabody, triabody, tetrabody, Bis-scFv, minibody, Fab2, Fab3 fragment, any combinations, hybrids or fused forms thereof. A Fc fragment comprises the carboxy-terminal portions of both H chains held together by disulfides. The effector functions of antibodies are determined by sequences in the Fc region; this region is also the part recognized by Fc receptors (FcR) found on certain types of cells. A Fv fragment comprises a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigens, although often at a lower affinity than the entire binding site. The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains.

In another example, the bridging molecule is a one-armed antibody. The term “one-armed antibody” or “one-armed antibodies” refers to an antibody that comprises (1) a variable domain joined by a peptide bond to a polypeptide comprising a CH2 domain, a CH3 domain or a CH2-CH3 domain, and (2) a second CH2, CH3 or CH2-CH3 domain, wherein a variable domain is not joined by a peptide bond to a polypeptide comprising the second CH2, CH3 or CH2-CH3 domain. In one embodiment, the one-armed antibody comprises 3 polypeptides (1) a first polypeptide comprising a variable domain (e.g., VH), CH1, CH2 and CH3, (2) a second polypeptide comprising a variable domain (e.g., VL) and a CL domain, and (3) a third polypeptide comprising a CH2 and CH3 domain. In an embodiment, the third polypeptide does not comprise a variable domain. In another embodiment, the one-armed antibody has a partial hinge region containing the two cysteine residues which form disulfide bonds linking the constant heavy chains. In one embodiment, the variable domains of the one armed antibody form an antigen binding region. In another embodiment, a variable domain of the one armed antibody is a single variable domain, wherein each single variable domain is an antigen binding region.

Antibodies or fragments herein may be “chimeric” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, provided that they exhibit the desired biological activity. Chimeric antibodies of interest herein include primatized antibodies comprising variable domain antigen-binding sequences derived from a non-human primate (e.g., Old World Monkey, Ape, etc.) and human constant region sequences.

In one example, a bridging molecule is a Fab polypeptide with two or more variable regions. In another example, a bridging molecule may be two Fab polypeptides linked together. In another example, a bridging molecule may be a nucleic acid comprising two more aptamers (e.g., with different binding affinity and/or specificity). In another example, a bridging molecule may be a nanobody or multiple nanobodies fused or liked together. In another example, a bridging molecule may be an antigen domain polypeptide or multiple antigen domain polypeptides fused together. In another example, a bridging molecule may be one or more Fc fragment polypeptides fused together.

In another example, a bridging molecule is a single chain variable fragment (scFv). As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::VL heterodimer. A scFV may be an antibody fragment that comprises the V_{H} and V_{L} antibody domains connected into a single polypeptide chain. In some cases, sFv polypeptide further comprises a polypeptide linker between the V_{H} and V_{L} domains, which enables the sFv to form the desired structure for antigen binding. The heavy (V_{3/4}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin.

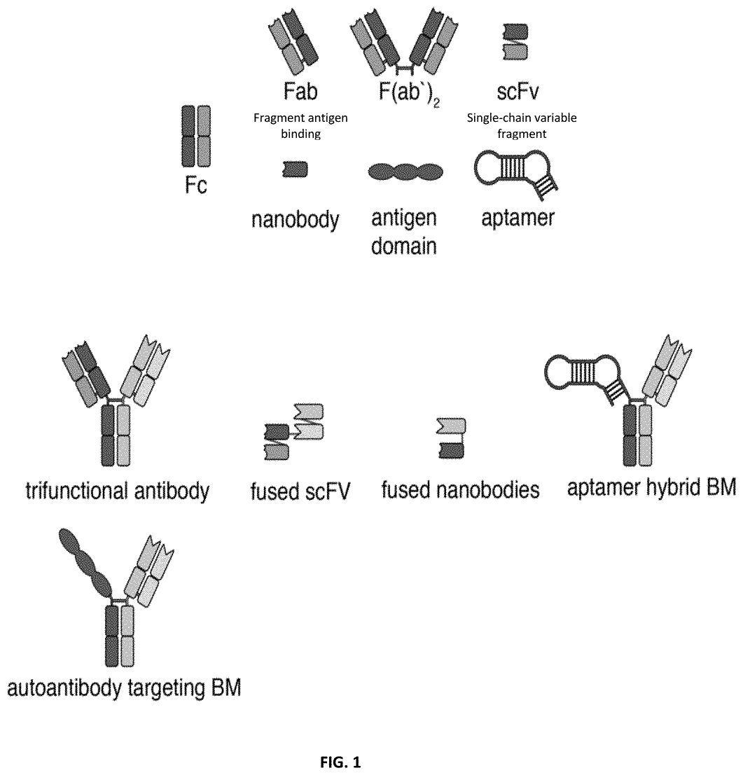

A bridging molecule may comprise any combination or hybrid of the binding components herein. FIG. 1 shows examples of the bridging molecule designs that can be used.

### Linkers

A bridging molecule may comprise two more binding components linked by a linker. A linker refers to any form of covalent or non-covalent attachment between two moieties (e.g., two binding components). An example of a linker is a peptide linker in a fusion protein. Chemical coupling or conjugation between the moieties is expressly included. In some cases, the binding components need not be linked directly, and linkage may occur via a linker peptide. The linker peptide may be a flexible or a structured linker peptide. Suitable flexible linker peptides may comprise one or more glycine residues, optionally in combination with other amino acid residues. A structured linker may comprise one or more proline residues and may comprise a defined secondary structure. Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond).

In some cases, a linker is an amino acid sequence of two or more amino acids in length. The linker may comprise neutral polar or nonpolar amino acids. A linker may be, for example, 2 to 100 amino acids in length, such as between 2 and 50 amino acids in length, for example, 3, 5, 10, 15, 20, 25, 30, 35, 40, 45, or 50 amino acids in length. A linker can be “cleavable,” for example, by auto-cleavage, or enzymatic or chemical cleavage. Cleavage sites in amino acid sequences and enzymes and chemicals that cleave at such sites are well known in the art and are also described herein. In some cases, the linkers may be peptide linkers, the sequences of which adopt a flexible extended conformation and do not exhibit a propensity for developing an ordered secondary structure. In certain embodiments, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180. For example, GlySer linkers GGS, GGGS or GSG can be used. GGS, GSG, GGGS or GGGGS linkers can be used in repeats of 3 (such as (GGS)₃ (SEQ ID NO: 11), (GGGGS)₃) (SEQ ID NO: 1) or 5, 6, 7, 9 or even 12 (SEQ ID NOs: 3, 4, 5, 7 or 13.) or more, to provide suitable lengths. In particular embodiments, linkers such as (GGGGS)₃ (SEQ ID NO: 1) are preferably used herein. (GGGGS)₆ (SEQ ID NO: 4) (GGGGS)₉ (SEQ ID NO: 7) or (GGGGS)₁₂ (SEQ ID NO: 13) may preferably be used as alternatives. Other preferred alternatives are (GGGGS)₁ (SEQ ID NO: 10), (GGGGS)₂ (SEQ ID NO: 14), (GGGGS)₄ (SEQ ID NO: 2), (GGGGS)₅ (SEQ ID NO: 3), (GGGGS)₇ (SEQ ID NO: 5), (GGGGS)₈ (SEQ ID NO: 6), (GGGGS)₁₀ (SEQ ID NO: 8), or (GGGGS)₁₁ (SEQ ID NO: 9).

In some embodiments, the linker is an oligonucleotide linker. An oligonucleotide linker may be single-stranded, fully double-stranded, or partially double-stranded. An oligonucleotide linker may be any length. For example, a oligonucleotide linker may be from 1 nucleotide to about 100 nucleotides in length. When the oligonucleotide linker is double-stranded, the linker can comprise a double stranded region of about 5 to about 500 consecutive base pairs. In some instances, the duplex region may be interrupted by one or more single-stranded regions in one or both of the strands of the duplex. Further, a double-stranded oligonucleotide linker may comprise a single-stranded overhang on one or both ends of the double-stranded region. Moreover, a oligonucleotide linker may comprise one or more nucleic acid modifications. A nucleic acid linker can be attached to a compound by a non-nucleic acid linker.

Additional examples of linkers include spacer molecules, selected molecules capable of attaching two aptamers (e.g., joining the two aptamers to form an aptameric multimolecular complex or synthetic heteropolymer), nonnucleotide dendrimers, dendrons, peptides, proteins, nonnucleotide linkages and bridges, nonnucleotide monomers, dimers and polymers, ligands (e.g., biotin, digoxigenin, FITC, DNP and peroxidase) and receptors (e.g., avidin, streptavidin and anti-digoxigenin, anti-FITC, anti-DNP and anti-peroxidase antibodies), lipids, sugars, polyethylene glycols, cholesterol, fusion proteins, bispecific antibodies, chelating agents, intercalating agents, crosslinking agents, and nonnucleotide molecules comprising bifunctional, heterofunctional and multifunctional molecules and oligonucleotide linkers. Additional useful linkers include: (i) EDC (1-ethyl-3-(3-dimethylamino-propyl)carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio)propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS(N-hydroxysulfo-succinimide: Pierce Chem. Co., Cat. #24510) conjugated to EDC.

In some embodiments, the bridging molecule may be a fusion protein comprising two or more binding components. Such a fusion protein may be expressed by a single DNA molecule. For example, the fusion protein may be a recombinant fusion protein. The terms, “fused” or “fusion” are used interchangeably. These terms refer to the joining together of two more elements or components by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

### Cells Bound by the Binding Components

A binding component may be any molecule that binds to a cell, e.g., via binding to a molecule on the cell. In some cases, one or more of the multiple binding components bind to a diseased cell and one or more of the multiple binding components bind to an therapeutic cell, e.g., an immune cell. The binding components may recruit the therapeutic cell to destruct the diseased cell.

### Diseased Cells

The binding components herein may bind to diseased cells. A diseased cell may be a cell in or derived from a diseased subject. A diseased cell may be in a diseased tissue or organ in the diseased subject. In some examples, a diseased cell may be a cell in a stage that diverges from the normal or healthy state and may result from the influence of a pathogen, a toxic substance, irradiation or cell internal deregulation. In some examples, a diseased cell may also be a cell that has been infected with a pathogen, e.g., a bacteria or viruses.

### Tumor Cells

A diseased cell may be a cell of a tumor. The cell of a tumor may be from or derived from the tumor. Examples of the tumor include liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma. Examples of tumors also include solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumor including serous tumor, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids, and Lymphoproliferative disorders.

The diseased cells may be associated with diseased tissues in patients with cancers, including breast cancer, colon cancer, lung cancer, prostate cancer, testicular cancer, brain cancer, skin cancer, rectal cancer, gastric cancer, esophageal cancer, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers. For example, a diseased cell may be a malignant cell or neoplastic cell that may constitute or give rise to cancer in an individual.

A diseased cell may comprise (e.g., express) one or more tumor-specific antigens. The term “neoantigen” or “tumor specific antigens” means a class of tumor antigens that arises from a tumor-specific mutation(s) which alters the amino acid sequence of genome encoded proteins. Examples of tumor-specific antigens include KG2D, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAB, WT-1, PSMA, NY-ESO1, AFP, CEA, CTAG1B, CD 19, CD33, α-Folate Receptor, CAIX, CD19, CD19/CD20, CD20, CD22, CD30, CD33, CD44v7/8, CD138, CD244, CEA, CS1, EBNA3C, EGP-2, EGP-40, EpCAM, rb-2, Erb-B 2, 3, 4, FBP, Fetal Acetylcholine Receptor, GD2, GD3, GPA7, Her2, Her2/new, IL-13R-a2, KDR, k-light chain, LeY, L1 Cell Adhesion Molecule, MAGE-A1, Mesothelin, MUC1, NKG2D Ligands, Oncofetal Antigen (h5T4), PSCA, PSMA, TAA Targeted by mAb IgE, TAG-72, VEGF-R2, and mutant ERBB2-interacting protein, and fragments thereof. In some examples, the diseased cells are melanoma tumor cells comprising an NY-eso-1 antigen. In some examples, the diseased cells are metastatic cholangiocarcinoma cells comprising mutant ERBB2-interacting protein antigen.

Additional examples of the antigens on tumor cells include B cell maturation antigen (BCMA) (see, e.g., Friedman et al., Effective Targeting of Multiple BCMA-Expressing Hematological Malignancies by Anti-BCMA CART Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CART Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostate; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDG1cp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis (Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDG1cp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27 m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

### Diseased Cells Involved in Autoimmune Diseases

The diseased cell herein may be cells associated with an autoimmune disease. In some embodiments, the diseased cell may be a cell involved in production and function of an autoantibody (e.g., autoantibody causing an autoimmune disease). For example, the diseased cell may be an immune cell. The immune cell may recognize a self-antigen, thus targeting the cells, tissues, or organ of the host and leading to autoimmune diseases.

The diseased cell may target (e.g., destroy or inhibit the activity or function of) a cell expressing a self-antigen. The term “self-antigen” refers to an antigen that originates from within a subject, or tissue, organ, or cell in the subject. In some embodiments, a self-antigen comprises an endogenous antigen. In some embodiments, self-antigens comprise neo-self-antigens, microbially or parasite encoded neo-self-antigens, or other neo-self-antigens expressed as a result of genetic alteration to an animal or cell. In some cases, the self-antigen may be an auto-antigen. The term “auto-antigen” refers to an antigen that comprises an epitope of a self-antigen or an immunologically reactive epitope that mimics that of a self-antigen. In some embodiments, the term auto-antigen comprises antigens to which autoantibodies are produced. In some embodiments, an auto antigen comprises an endogenous antigen wherein the animal from which the endogenous antigen originated is or was once immunologically tolerant to the selected antigen.

In some examples, a diseased cell is in a subject with an autoimmune disease and targets an self-antigen associated with the autoimmune disease. Examples of autoimmune diseases include Celiac disease, Hashimoto's thyroiditis, Autoimmune thrombocytopenic pupura, Goodpasture's syndrome, Pemphigus vulgaris, Acute rheumatic fever, Bullous pemphigoid, Mixed connective tissue disease, Guillain-Barré syndrome, Neuromyelitis optica, diabetes mellitus (e.g., type I diabetes), arthritis (including rheumatoid arthritis, juvenile rheumatoid arthritis, ostecarthritis, psoriasic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing haemorrhagic encephalopathy, idiopathic bilateral progressive sensorineural hearing loss, aplastic anaemia, pure red cell anaemia, idiopathic thrombocytopenia, polychondritis, Wegener's granulomatosis, chronic active hepatitis, Stevens-Johnson syndrome, idiopathic sprue, lichen planus, Graves ophthalmopathy, grace's disease sarcoidosis, primary biliary cirrhosis, uveitis posterior, interstitial lung fibrosis, polymyositis, dermatomyositis, interstitial lung disease, Raynaud's phenomenon, also scleroderma (PM-scl), autoimmune gastritis, type A chronic atrophic gastritis, pernicious anaemia, primary myxedema, subacute thyroiditis, myocarditis and cardiomyopathy and related diseases, Felty's syndrome/autoimmune, neutropenia, systemic lupus erythematosus, subacute systemic lupus erythematosus, Addison's Disease, types I and II, autoimmune polyglandular syndrome (APS), hypoparathyroidism, vitiligo celiac disease, gluen-sensitive enteropathy, inflammatory arthritis/rhewnatoid arthritis, autoimmune hemolytic anemia, various connective tissue diseases, various hepatic and connective tissue autoimmune diseases, autoimmune thrombocytopenia, purpura, various autoimmune hepatitis and primary biliary cirrhosis (UGT-1 and mitochondrial enzymes), Stiff-Man syndrome, Rasmussen's encephalitis, Guillain-Barre Syndrome, and related neuronal syndromes (e.g., Miller-Fisher Syndrome); and autoimmune diabetes (sulphatide). Other autoantigens include those derived from nucleosomes for the treatment of systemic lupus erythematosus.

Diseased cells may include cells that target, recognize, and bind to self-antigens of other cells. Examples of self-antigens include a lupus autoantigen, Smith, Ro, La, U1-RNP, fibrillin (scleroderma), pancreatic β cell antigens, GAD65 (diabetes related), insulin, myelin basic protein, myelin proteolipid protein, histones, PLP, collagen (e.g., type I collagen, type II collagen), glucose-6-phosphate isomerase, citrullinated proteins and peptides, thyroid antigens, thyroglobulin, thyroid-stimulating hormone (TSH) receptor, various tRNA synthetases, components of the acetyl choline receptor (AchR), MOG, proteinase-3, myeloperoxidase, epidermal cadherin, acetyl choline receptor, platelet antigens, nucleic acids, nucleic acid:protein complexes, joint antigens, antigens of the nervous system, salivary gland proteins, skin antigens, kidney antigens, heart antigens, lung antigens, eye antigens, erythrocyte antigens, liver antigens, stomach antigens, T cell responses and B cell responses with the production of antibodies to nucleosomes, splicing ribonucleoproteins, DNA, RNA, and Sm antigens in Systemic Lupus Erythmatosus (Casciola-Rosen et al, 1994), Topoisomerase 1 (alpha Topoisomerase 1), centromere antigens (e.g., in Scleroderma (Casciola-Rosen, 1997)), RNP antigens in Mixed Connective Tissue Disease Sharp, 1977), aminoacyl tRNA in Myositis (Tan, 1992), antibodies to thyroid microsomal peroxidase and thyroglobulin antigens in Thyroid Disorders (Rose and McKay, 1985), and islet cell insulin and GAD65 antibodies in Diabetes Mellitus (Wucherpfennig, 2001) and to IgG as Rheumatoid Factors in Rheumatoid Arthritis, myelin basic protein (MBP), proteolipid protein, myelin oligodendrocyte glycoprotein, αβ-crystallin, myelin-associated glycoprotein, Po glycoprotein, PMP22, 2′,3′-cyclic nucleotide 3′-phosphohydrolase (CNPase), glutamic acid decarboxylase (GAD), insulin, 64 kD islet cell antigen (IA-2, also termed ICA512), phogrin (IA-2β), type II collagen, human cartilage gp39 (HCgp39), gp130-RAPS, acetylcholine receptor (AChR), titin (connectin), neuronal voltage-gated calcium channel, CNS myelin-basic-protein (MBP), MBP₈₃₋₉₉ epitope, proteolipid protein (PLP), PLP₁₃₉₋₁₅₁ epitope, PLP₁₇₈₋₁₉₁ epitope, myelin oligodendrocyte glycoprotein (MOG), MOG₉₂₋₁₀₆ epitope, ai3-crystallin, myelin-associated glycoprotein (MAG), Po glycoprotein and PMP22, 2′,3′-cyclic nucleotide 3′phosphohydrolase (CNPase), glutamic acid decarboxylase (GAD), and isoforms thereof (e.g., 65 and 67 kDa isoforms), 64 kD islet cell antigen/tyrosine phosphatase-like islet, cell antigen-2 (IA-2, also termed ICA512), phogrin (IA-213), fibrillarin, U3-small nuclear protein (snoRNP), Jo-1 antigen/aminoacyl histidyl-tRNA synthetase, PL-7 antigen/threonyl tRNA synthetase, PL-12 antigen/alanyl tRNA synthetase, EJ antigen/glycyl-tRNA Synthetase, OJ antigen/NJ antigen, isoleucyl-tRNA synthetase, signal recognition particle (SRP), Mi-2 helicase, PM-scl proteins (75 kDa, 100 kDa), KJ antigen, Fer antigen/elongation fractor 1a, Mas antigen/tRNASer, type IV collagen a3 chain, Smith (Sm) antigens and snRNP's, including snRNPs, D1, D2, D3, B, B′, B3 (N), E, F, and G, as found in RNP complexes U1, U2, U4/6, and U5, nRNP U1-snRNP complex including subunits U1-70 kD, A and C, deoxyribonucleic acid (DNA) double-stranded B-form, deoxyribonucleic acid (DNA) denatured/single-stranded, CyclinA, Ro (SS-A) antigens (e.g., 52 kDa and 60 kDa), La (SS-B) antigen, proteinase-3 (serine proteinase)/cytoplasmic, neutrophil antigen (cANCA)/myeloblastin, myeloperoxidase/nuclear or perinuclear neutrophil antigen (pANCA), 13r glycoprotein-1 (aka apolipoprotein H), cardiolipin, phosphatidylcholine, and various anionic phospholipids, parietal cell antigen; H•/KATPase gastric proton pump a & 13 subunits, thyroglobulin (TG); TG₁₁₄₉₋₁₂₅₀, thyroid peroxidase (TPO) (e.g., TPO₅₉₀₋₆₇₅ and TPO₆₅₁₋₁₅₀), thyroid-stimulating hormone receptor (TSH-R, also termed thyrotropin), desmosomal proteins; desmoglein-1, desmoglein-3, hemidesmosome proteins, BP180 (also known as BPAG2 and type XVII collagen) and BP230 (BPAG1), type VII collagen, mitochondrial pyruvate dehydrogenase complex (PDC), E1a decarboxylase, mitochondrial E 1β decarboxylase, mitochondrial PDC-E2 acetyltransferase, mitochondrial protein X, mitochondrial branched chain, 2-oxo acid dehydrogenase (BCOADC) E2 subunit, PDC-E2 (mitochondrial pyruvate dehydrogenase dihydrolipoamide acetyltransferase), 2-oxoglutarate dehydrogenase (OGDC); E2 succinyl transferase, chromosomal centromere proteins CENP-A, B, C and F coilin/p80, HMG proteins (e.g., HMG-1, HMG-2, HMG-14, HMG-17), Histone proteins (e.g., H1, H2A, H2B, H3 and H4), Ku antigen (p70/p80) and DNA-PK catalytic subunit, NOR-90/hUBF, Proliferating cell nuclear antigen (PCNA ribosomal RNP proteins (“Pantigens”), PO, P1 and P2, Ra33/hnRNP A2, SP-100, S-antigen/interphotoreceptor, retinoid binding protein (IRBP), annexinXl (56K autoantigen), hair follicle antigens, human tropomyosin isoform 5 (hTM5), cardiac myosin, larminin, β1-adrenergic receptors, mitochondrial adenine nucleotide translocator (ANT), mitochondrial branched-chain ketodehydrogenase (BCKD), eukaryotic elongation factor 1A-1 (eEF1A-1), glycoprotein gp70 (viral antigen), early endosome antigen-1 (EEA1), 21-hydroxylase, calcium sensing receptor (CaSR), tissue transglutaminase (e.g., tyrosinase tissue transglutaminase), keratin proteins, poly (ADP-ribose) polymerase (PARP), nucleolar proteins B23/numatrin, erythrocyte surface antigens/glycophorins, RNA polymerase I subunits, RNA polymerase II subunits, RNA polymerase III subunits, Th/To (7-2 RNP; also known as RNase MRP), nuclear mitotic apparatus proteins (NuMA proteins), nuclear lamins A, B and C 210-kDa glycoprotein (gp210), pericentriolar material protein 1 (PCM-1), platelet surface antigens/glycoproteins IIb/Illa and 1b/IX, golgins (e.g., 95 and 160-kDa species), F-actin, cytochrome P-450 superfamily, proteins, most specifically 2D6; epitopes: 2D6₂₅₇₋₂₆₉, 2D6₃₂₁₋₃₅₁, 2D6₃₇₃₋₃₃₉, and 2D6₄₁₉₋₄₂₉, P-450 proteins 1A2, 2B, 2C9, 2C11, 2E, 3A1, c21, sec, and c 17a, UDP-glucuronosyltransferase, family proteins (UGT-1 and UGT-2), asialoglycoprotein receptor (ASGP-R), amphiphysin glutamate receptor Glu R3, human gangliosides, especially GM1, and also GD1a, Nacetylgalactosaminyl-GD1a, GD1b, GQb1, GQ1b, GD3, LM1, GT1a, GT1b and asialo-GM1, and sulphatide (3′-sulphogalactosylceramide), Mitochondrial antigens, Rheumatoid factor, cycle citrullinated peptide, tyrosine phosphatase-like protein, Platelet integrin, GpIIb:IIIa, Non-collagenous domain of basement membrange collagen type IV, Streptococcal cell-wall antigens, Type XVII collagen, Dystonin, AQP4, amyloid beta, amyloid precursor protein, collagen, sodium-iodide symporter, and any combination thereof.

The diseased cells may be immune cells described herein that target cells with self-antigens. Examples of such diseased cells include T cells and B cells targeting self-antigens. In some examples, a diseased cell may be an autoimmune B cell. In some cases, the binding component may be recognizable by an autoantibody. For example, the binding component may comprise an antigen recognized by an autoantibody.

In some examples, the diseased cell is in a subject with Celiac disease and targets tissue transglutaminase. In some examples, the diseased cell is in a subject with Hashimoto's thyroiditis and targets Thyroid peroxidase. In some examples, the diseased cell is in a subject with Graves' disease and targets TSH receptor. In some examples, the diseased cell is in a subject with Primary biliary cirrhosis and targets Mitochondrial antigens. In some examples, the diseased cell is in a subject with Rheumatoid arthritis and targets Rheumatoid factor and/or cycle citrullinated peptide. In some examples, the diseased cell is in a subject with Scleroderma and targets Centromere antigen and/or topoisomerase I. In some examples, the diseased cell is in a subject with Sjogren's syndrome and targets Ro and La antigens. In some examples, the diseased cell is in a subject with Type I diabetes and targets Insulin, glutamic acid decarboxylase, and/or tyrosine phosphatase-like protein. In some examples, the diseased cell is in a subject with Autoimmune thrombocytopenic pupura and targets Platelet integrin GpIIb:IIIa. In some examples, the diseased cell is in a subject with Goodpasture's syndrome and targets Non-collagenous domain of basement membrane collagen type IV. In some examples, the diseased cell is in a subject with Pemphigus vulgaris and targets Desmoglein 1 and/or desmolgein 3. In some examples, the diseased cell is in a subject with Acute rheumatic fever and targets Streptococcal cell-wall antigens. In some examples, the diseased cell is in a subject with Bullous pemphigoid and targets Type XVII collagen and/or Dystonin. In some examples, the diseased cell is in a subject with Multiple sclerosis and targets Myelin basic protein. In some examples, the diseased cell is in a subject with Mixed connective tissue disease and targets U1-RNP. In some examples, the diseased cell is in a subject with Guillain-Barré syndrome and targets GM1, GD1a, GT1a, GQ1b, and/or GD3. In some examples, the diseased cell is in a subject with Myasthenia gravis and targets Acetylcholine receptor. In some examples, the diseased cell is in a subject with Neuromyelitis optica and targets AQP4.

### Therapeutic Cells

One of the binding components may bind to therapeutic cells, which may destroy or inhibit the activity and/or function of the diseased cells bound by another binding component.

### Immune Cells

In some examples, the therapeutic cells may be immune cells. As used herein, the term “immune cell” is intended to include a cell which plays a role in specific immunity (e.g., is involved in an immune response) or plays a role in natural immunity. The immune cells may target and thus destroy the diseased cells herein. Examples of the immune cells include all distinct classes of lymphocytes (T lymphocytes, such as helper T cells and cytotoxic T cells, B lymphocytes, and natural killer cells), monocytes, macrophages, mast cells, macrophages, dendritic cells, natural killer cells, and other antigen presenting cells, dendritic cells, and leukocytes (e.g., neutrophils, eosinophils, and basophils).

In some examples, a binding component may bind to a T cell. The T cell may be helper T cell, cytotoxic T cell, memory T cell, regulatory T cell, natural killer T cell, gamma delta T cell, or mucosal associated invariant T cell. In some examples, the immune cells are cytotoxic T cells, e.g., CD8+ T cells. some examples, the immune cells are helper T cells, e.g., CD4+ helper T cells.

In certain cases, a bridging molecule may comprise a binding component that binds to a T cell and another binding component that binds to an accessory cell. Accessory cells or antigen presenting cells include nonlymphoid cells such as macrophages, dendritic cells, and Langerhans cells (epithelial dendritic cells), natural killer cells that function to present antigens to MHC-restricted T cells. They may not be antigen specific and may present different costimulatory molecules, either soluble cytokines or membrane bound peptides, with the antigen to the T-cells. The specific combination of antigen, MHC-restricted T cell, and expressed costimulatory molecules may determine the specific immune response. In certain cases, a bridging molecule may further comprise a third binding component that binds to a diseased cell.

### Engineered Cells

The therapeutic cells used herein may be cells administered to the subject receiving the bridging molecule. Such cells may be engineered therapeutic cells described herein. For example, the engineered therapeutic cells may recognize one or more of the antigens herein. In some embodiments, the bridging molecule may facilitate cell therapy by bringing engineered cells to diseased cells. In some examples, the engineered cells may be T cells that are supplied to the patient with a known orthogonal antigen that the bridging molecule may then target with a binding component. This may be advantageous, because this interaction may be highly specific and may not interfere with other endogenous processes. Additionally, the T cells may then be engineered to be more active or have other functionalities to increase therapeutic potential.

The engineered T may express a desired receptor. Such cells may be engineered to express a desired receptor. In some examples, the engineered cells may be chimeric antigen receptor (CAR) T cells. In some embodiments, the CAR T cells may be those described in paragraphs [0095]-[0171] of WO2018191553A1.

### Pharmaceutical Composition

The present disclosure also provides for pharmaceutical compositions comprising one or more bridging molecules. A “pharmaceutical composition” refers to a composition that usually contains an excipient, such as a pharmaceutically acceptable carrier that is conventional in the art and that is suitable for administration to cells or to a subject.

In some embodiments, the methods of the disclosure include administering to a subject in need thereof an therapeutically effective amount (e.g., therapeutically effective amount or prophylactically effective amount) of bridging molecule(s) provided herein.

The term “therapeutic effect” or “pharmaceutically effect” refers to some extent of relief of one or more of the symptoms of a disorder (e.g., a neoplasia or tumor) or its associated pathology. “Therapeutically effective amount” or “pharmaceutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” or “pharmaceutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

The administration of the pharmaceutical compositions may be supplemented with other known treatments, such as surgery on the subject. In certain embodiments, the surgery is strictureplasty, resection (e.g., bowel resection, colon resection), colectomy, surgery for abscesses and fistulas, proctocolectomy, restorative proctocolectomy, vaginal surgery, cataract surgery, or a combination thereof.

In some cases, the pharmaceutical compositions further comprise pharmaceutically acceptable carrier(s) and/or excipient(s). The term “pharmaceutically acceptable” as used throughout this specification is consistent with the art and means compatible with the other ingredients of a pharmaceutical composition and not deleterious to the recipient thereof. As used herein, “carrier” or “excipient” includes any and all solvents, diluents, buffers (such as, e.g., neutral buffered saline or phosphate buffered saline), solubilizers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition may be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation may be administered on a support, scaffold, matrix or material to provide improved tissue regeneration. For example, the material can be a granular ceramic, or a biopolymer such as gelatin, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

The pharmaceutical compositions may comprise one or more pharmaceutically acceptable salts. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methyl sulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agents, cells, agonists, antagonists, antibodies or fragments thereof to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved, for example, and not by way of limitation, by local infusion during surgery, topical application, by injection, by means of a catheter, by means of a suppository, or by means of an implant.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

### Methods of Treatment

The present disclosure further provides for methods of treating or preventing a disease. In general, such methods may comprise administering one or more bridging molecules or a pharmaceutical composition comprising such bridging molecule(s) to a subject in need thereof.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treat,” “treated,” “treating,” “treatment,” and the like are meant to refer to reducing or ameliorating a disorder and/or symptom associated therewith (e.g., a neoplasia or tumor). “Treating” may refer to administration of the therapy to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The bridging molecule may be used for treating cancer. In some examples, while there are known neo-antigens for some cancers, an example clinical approach may be to sequence a patient's tumor and identify neo-antigens for that are unique to a given patient. Because the space of potential neoantigens may be bounded, eventually an entire class of bridging molecules may be made for each type of cancer, with specific combinations formulated for a given patient's neo-antigen repertoire. For autoimmune diseases, the antigen may either be fused directly to the bridging molecule of one of the chains, or the antigen may be targeted via a binding region on the bridging molecule. In some cases, by designing bridging molecules to target tumor antigens and form a bridge to native immune cells, tumor cells may be systematically lysed, regardless of whether native reactivity exists. In some cases, bridging molecules may be designed as a cocktail such that multiple neo-antigens are targeted from a given tumor.

The bridging molecule may be used for treating autoimmune diseases. In some examples, the bridging molecule may be designed to causes specific lysis of B cells/plasma cells responsible for perpetuating autoimmunity. A bridging molecule may be synthesized with specificity for the antigen involved, or via direct fusion of the antigen to the bridging molecule scaffold, which may then bind to the immunoglobulin on the B cell/plasma cell surface and cause directed lysis by recruitment of CD8+ T cells or accessory immune cells. In some cases, the methods are used for treating autoimmune diseases mediated by autoantibodies or B-cells of known self-antigen. In such cases, one of the Fab or binding regions of the bridging molecule may be replaced with the epitope region of the antigen. This fusion may specifically bind to the autoantibodies on the autoimmune B-cells and lead to their destruction via antibody-dependent cell-mediated cytotoxicity or T-cell mediated pathways.

The present application also provides aspects and embodiments as set forth in the following numbered Statements:

Statement 1. An engineered, non-naturally occurring molecule, comprising: (a) a first binding component capable of binding to a T cell; and (b) a second binding component capable of binding to a diseased cell.

Statement 2. The molecule of statement 1, wherein the first and the second binding components are selected from the group consisting of Fab fragment, single-chain variable fragment (scFv), nanobody, aptamer, antigen, and antigen-binding region.

Statement 3. The molecule of statement 1 or 2, wherein the first and the second binding components are Fab fragments recognizing different antigens.

Statement 4. The molecule of any one of statements 1-3, further comprising an Fc region that binds to Fc-gamma receptor positive cells.

Statement 5. The molecule of any one of statements 1-4, wherein the Fc-gamma receptor positive cells are macrophages, neutrophils, eosinophils, dendritic cells, or natural killer cells.

Statement 6. The molecule of any one of statements 1-5, wherein the first and the second binding components are scFvs and linked by a linker.

Statement 7. The molecule of any one of statements 1-6, wherein the first and the second binding components are nanobodies and linked by a linker.

Statement 8. The molecule of any one of statements 1-7, wherein the first binding component is a Fab fragment and the second binding component is an aptamer or a 10th type III fibronectin (Fn3) domain.

Statement 9. The molecule of any one of statements 1-8, wherein the first binding component is a Fab fragment and the second binding component is an antigen or a fragment thereof.

Statement 10. The molecule of statement 9, wherein the antigen is recognized by an autoantibody.

Statement 11. The molecule of statement 10, wherein the antigen is selected from the group consisting of tissue transglutaminase, thyroid peroxidase, TSH receptor, mitochondrial antigen, rheumatoid factor, cycle citrullinated peptide, centromere antigen, topoisomerase I, Ro and La antigens, RNP, Sm, dsDNA, cardiolipin, insulin, glutamic acid decarboxylase, tyrosine phosphatase-like protein, platelet integrin GpIIb:IIIa, non-collagenous domain of basement membrane collagen type IV, desmoglein 1, desmolgein 3, Streptococcal cell-wall antigen, type XVII collagen, dystonin, myelin basic protein, U1-RNP, GM1, GD1a, GT1a, GQ1b, GD3, acetylcholine receptor, and AQP4.

Statement 12. The molecule of any one of statements 1-11, wherein the T cell is a CD8+ T cell.

Statement 13. The molecule of any one of statements 1-12, wherein the T cell is a CD4+ T cell.

Statement 14. The molecule of any one of statements 1-13, wherein the diseased cell is a tumor cell.

Statement 15. The molecule of any one of statements 1-14, wherein the diseased cell is an autoimmune B cell.

Statement 16. A pharmaceutical composition comprising an engineered, non-naturally occurring molecule of any one of statements 1-15.

Statement 17. The pharmaceutical composition of statement 16, further comprising a pharmaceutically acceptable carrier or excipient.

Statement 18. A method of treating a disease, comprising administering a pharmaceutically effective amount of an engineered, non-naturally occurring molecule of any one of statements 1-15 to a subject in need thereof.

Statement 19. The method of statement 18, wherein the disease is a cancer.

Statement 20. The method of statement 18 or 19, wherein the cancer is selected from the group consisting of melanoma, and metastatic cholangiocarcinoma.

Statement 21. The method of any one of statements 18-20, wherein the disease is an autoimmune disease.

Statement 22. The method of any one of statements 18-21, wherein the autoimmune disease is selected from the group consisting of celiac disease, Hashimoto's thyroiditis, Graves' disease, primary biliary cirrhosis, rheumatoid arthritis, scleroderma, Sjogren's syndrome, SLE, type I diabetes, autoimmune thrombocytopenic pupura, Goodpasture's syndrome, Pemphigus vulgaris, acute rheumatic fever, bullous pemphigoid, multiple sclerosis, mixed connective tissue disease, Guillain-Barre syndrome, myasthenia gravis, and neuromyelitis optica.

Statement 23. The method of any one of statements 18-22, further comprising administering to the subject an engineered T cell.

Statement 24. The method of statement 23, wherein the engineered, non-naturally occurring molecule is capable of binding to the engineered T cell.

Statement 25. The use of engineered, non-naturally occurring molecule of any one of statements 1-15 or a pharmaceutical composition of statement 16 or 17 for the manufacture of a medicament for the treatment of a disease.

### EXAMPLES

### Example 1

To model destruction of autoimmune B cells, B cells from AM14 heavy chain (HC) mice are used. The B cells express autoantibodies targeting autologous IgG2a and present symptoms of rheumatoid arthritis. A bridging molecule is designed. The bridging molecule has the region of IgG2a containing the epitope for the AM14 B cells and combined with a CD3 targeting scFv. Upon co-administration of this antibody with CD8+ T-cells, cell-dependent death of the B cells is monitored.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered, non-naturally occurring molecule, comprising:
(a) a first binding component capable of binding to a T cell; and
(b) a second binding component capable of binding to a diseased cell.

2. The molecule of claim 1, wherein the first and the second binding components are selected from the group consisting of Fab fragment, single-chain variable fragment (scFv), nanobody, aptamer, antigen, and antigen-binding region.

3. The molecule of claim 1, wherein the first and the second binding components are Fab fragments recognizing different antigens.

4. The molecule of claim 1, further comprising an Fc region that binds to Fc-gamma receptor positive cells.

5. The molecule of claim 4, wherein the Fc-gamma receptor positive cells are macrophages, neutrophils, eosinophils, dendritic cells, or natural killer cells.

6. The molecule of claim 1, wherein the first and the second binding components are scFvs and linked by a linker.

7. The molecule of claim 1, wherein the first and the second binding components are nanobodies and linked by a linker.

8. The molecule of claim 1, wherein the first binding component is a Fab fragment, and the second binding component is an aptamer or a 10th type III fibronectin (Fn3) domain.

9. The molecule of claim 1, wherein the first binding component is a Fab fragment, and the second binding component is an antigen or a fragment thereof.

10. The molecule of claim 9, wherein the antigen is recognized by an autoantibody.

11. The molecule of claim 10, wherein the antigen is selected from the group consisting of tissue transglutaminase, thyroid peroxidase, TSH receptor, mitochondrial antigen, rheumatoid factor, cycle citrullinated peptide, centromere antigen, topoisomerase I, Ro and La antigens, RNP, Sm, dsDNA, cardiolipin, insulin, glutamic acid decarboxylase, tyrosine phosphatase-like protein, platelet integrin GpIIb:IIIa, non-collagenous domain of basement membrane collagen type IV, desmoglein 1, desmolgein 3, Streptococcal cell-wall antigen, type XVII collagen, dystonin, myelin basic protein, U1-RNP, GM1, GD1a, GT1a, GQ1b, GD3, acetylcholine receptor, and AQP4.

12. The molecule of claim 1, wherein the T cell is a CD8+ T cell.

13. The molecule of claim 1, wherein the T cell is a CD4+ T cell.

14. The molecule of claim 1, wherein the diseased cell is a tumor cell.

15. The molecule of claim 1, wherein the diseased cell is an autoimmune B cell.

16. A pharmaceutical composition, comprising: an engineered, non-naturally occurring molecule of claim 1.

17. The pharmaceutical composition of claim 16, further comprising a pharmaceutically acceptable carrier or excipient.

18. A method of treating a disease, comprising: administering a pharmaceutically effective amount of an engineered, non-naturally occurring molecule of claim 1 to a subject in need thereof.

19. The method of claim 18, wherein the disease is a cancer.

20. The method of claim 19, wherein the cancer is selected from the group consisting of melanoma and metastatic cholangiocarcinoma.

21. The method of claim 18, wherein the disease is an autoimmune disease.

22. The method of claim 21, wherein the autoimmune disease is selected from the group consisting of celiac disease, Hashimoto's thyroiditis, Graves' disease, primary biliary cirrhosis, rheumatoid arthritis, scleroderma, Sjogren's syndrome, SLE, type I diabetes, autoimmune thrombocytopenic pupura, Goodpasture's syndrome, Pemphigus vulgaris, acute rheumatic fever, bullous pemphigoid, multiple sclerosis, mixed connective tissue disease, Guillain-Barre syndrome, myasthenia gravis, and neuromyelitis optica.

23. The method of claim 18, further comprising administering to the subject an engineered T cell.

24. The method of claim 23, wherein the engineered, non-naturally occurring molecule is capable of binding to the engineered T cell.
